(19) United States
(12) Patent Application Publication
(10) Pub. No.: US 2011/0052687 A1
(43) Pub. Date: Mar. 3, 2011

(54) EXTENDED RELEASE PHARMACEUTICAL COMPOSITION OF PALIPERIDONE

(75) Inventors: Kamal Mehta, Bhilwara (IN); Pankaj Shinde, Jalgaon (IN); Hidaytulla Aga, Karad (IN); Rajadeep Gupta, Katni (IN)

(73) Assignee: GLENMARK GENERICS LTD, Mumbai (IN)

(21) Appl. No.: 12/862,988

(22) Filed: Aug. 25, 2010

(30) Foreign Application Priority Data
Aug. 26, 2009 (IN) 1960/MUM/2009
Aug. 18, 2010 (IN) 2322/MUM/2010

(51) Int. Cl. A61K 9/24; A61K 9/00; A61K 31/519; A61P 25/00

(52) U.S. Cl. 424/472; 424/400; 514/259.41

(57) ABSTRACT

The present invention provides an extended release tablet of paliperidone, comprising a) a core containing paliperidone and at least one water soluble and/or gellable polymer, b) a coating comprising at least one water insoluble or permeable polymer, and a water soluble and/or gellable polymer and optionally an enteric polymer.

### PRIORITY

This application claims priority to Indian Provisional Applications No. 1960/MUM/2009, filed on Aug. 26, 2009 and 2322/MUM/2010 filed on Aug. 18, 2010, the contents of which are hereby incorporated by reference.

### FIELD OF INVENTION

The present invention relates to an extended-release pharmaceutical composition comprising paliperidone or a pharmaceutically acceptable salt thereof; and a process for its preparation.

### BACKGROUND OF INVENTION

Paliperidone, a psychotropic agent of the chemical class of benzisoxazole derivatives, is used for the acute and maintenance treatment of schizophrenia. Commercially available INVEGA® tablets consist of paliperidone in an extended-release form. The commercially available tablet uses a delivery system that works on osmotic pressure to deliver paliperidone at a controlled rate. The osmotic pump devices are also described in patent applications, U.S. Patent Application No. 2004/0092534 and U.S. Patent Application No. 2007/0190137, where these disclose once-a day capsule shaped tablet of paliperidone that allows the release of paliperidone in ascending rate

U.S. Patent Application No. 2006/034927 describes a sustained release composition of paliperidone comprising a delay layer comprising (i) a polymeric matrix, and (ii) microencapsulated paliperidone, wherein the delay layer is substantially free of non-microencapsulated paliperidone; and a second layer comprising (iii) a polymeric matrix, and (iv) non-microencapsulated paliperidone matrix; wherein the second layer is located adjacent to the delay layer.

U.S. Patent Application No. 2009/087487 relates to a sustained release dosage form of Paliperidone comprising: two components system wherein the first component comprises at least one delay layer comprising a polymer, and the second component comprises non-coated paliperidone.

The present invention provides an extended-release tablet composition of paliperidone or pharmaceutically acceptable salt thereof, more particularly non-osmotic pump composition, which is suitable for once-daily oral administration.

### SUMMARY OF THE INVENTION

The present invention provides an extended release tablet of paliperidone, comprising a core tablet comprising paliperidone and at least one water soluble and/or gellable polymer, and a coating comprising at least one water insoluble or permeable polymer.

The present invention provides an extended release tablet of paliperidone, comprising a) core containing paliperidone and at least one water soluble and/or gellable polymer, b) a coating comprising at least one water insoluble or permeable polymer, and water soluble and/or gellable polymer and optionally an enteric polymer.

The present invention provides an extended release tablet of paliperidone, comprising a) core containing paliperidone and at least one water soluble and/or gellable polymer, b) a coating comprising at least one water insoluble or permeable polymer, and a water soluble and/or gellable polymer c) a coating comprising an enteric polymer.

In a further preferred embodiment, the present invention provides an extended release composition of paliperidone, comprising a) a core component comprising paliperidone and at least one water soluble and/or gellable polymer b) a first coat comprising a water insoluble or permeable polymer and a water soluble and/or gellable polymer c) a second coat comprising water insoluble or permeable polymer and a water soluble and/or gellable polymer, wherein the water insoluble or permeable polymer and water soluble and/or gellable polymer of first coat and second coat are the same, however the ratio of the water insoluble or permeable polymer and water soluble and/or gellable polymer in second coat is different from that of the first coat.

The present invention further provides a method for the preparation of the extended release composition comprising paliperidone or pharmaceutically acceptable salts thereof.

### DETAILED DESCRIPTION OF INVENTION

The commercially available tablets of paliperidone use a delivery system that works on osmotic pressure to deliver paliperidone at a controlled rate. The delivery system, in the semblance of a capsule-shaped tablet, consists of an osmotically active trilayer core surrounded by a subcoat and semipermeable membrane. The trilayer core is composed of two drug layers containing the drug and excipients, and a push layer containing osmotically active components. There are two precision laser-drilled orifices on the drug-layer dome of the tablet. In an aqueous environment, such as the gastrointestinal tract, the water-dispersible overcoat quickly erodes. Water then enters the tablet through the semi-permeable membrane that controls the rate at which water enters the tablet core, which, in turn, determines the rate of drug delivery. The hydrophilic polymers of the core hydrate and swell, creating a gel containing paliperidone that is then pushed out through the tablet orifice.

The osmotic pump devices are also described in patent applications, U.S. Patent Application No. 2004/0092534 and U.S. Patent Application No. 2007/0190137, where these disclose once-a day capsule shaped tablet of paliperidone that allows the release of paliperidone in ascending rate. Said tablet consist of a semi-permeable membrane surrounding a three layer core wherein the first layer contains low amounts of drug and an osmotic agent; the middle layer contains higher amounts of drug and without osmotic agent and the third layer is a push layer.

U.S. Patent Application No. 2006/034927 describes a sustained release composition of paliperidone comprising a delay layer comprising (i) a polymeric matrix, and (ii) microencapsulated paliperidone, wherein the delay layer is substantially free of non-microencapsulated paliperidone; and a second layer comprising (iii) a polymeric matrix, and (iv) non-microencapsulated paliperidone matrix; wherein the second layer is located adjacent to the delay layer.

U.S. Patent Application No. 2009/087487 relates to a sustained release dosage form of paliperidone comprising: two component systems, wherein the first component comprises at least one delay layer comprising a polymer, and the second component comprises non-coated paliperidone.

The present invention provides an extended-release tablet composition of paliperidone or pharmaceutically acceptable salt thereof, more particularly non-osmotic pump composition, which is suitable for once-daily oral administration.

The phrase, “extended release compositions”, as used herein, is intended to mean non-osmotic pump compositions that release paliperidone for several hours, preferably up to about 24 hours.

The phrase, “water soluble and/or gellable polymer” as used herein, is intended to encompass a selection from the group comprising of methyl cellulose, hydroxypropyl methylcellulose, hydroxyethylcellulose, hydroxypropyl cellulose, hydroxyethylmethylcellulose, carboxymethylcellulose and sodium carboxymethylcellulose; polyvinylpyrrolidone, polyethylene oxide; and polysaccharides. A preferred polymer is hydroxypropyl methylcellulose (HPMC) and derivatives thereof. HPMC derivatives are available in a low, normal or high viscosity grades. The rate of hydration and extent of gelling capacity of polymer controls a release rate of the drug from the formulation. Specific HPMCs which are most suitable are Methocel™ K100M, K15M, F4M, E4M, K4M, K100LV, K3, E15LV, E15LN, E15CLV, E50, E5 and E3 (available from Dow Chemical, Midland Mich.).

The present invention presents preferably the core tablet, which is common for all aspects of the present invention, comprising paliperidone and water soluble and/or gellable polymer such as, hydroxypropylmethylcellulose at about 10% to about 40%, preferably about 15% to about 35% by weight of an uncoated core. Hydroxypropylmethylcellulose can be used alone; more preferably, the combination of two or more hydroxypropylmethylcelluloses of different gelling capacities. For the purpose of core tablet of the present invention, hydroxypropylmethylcelluloses of a viscosity more than 100 cps, such as Methocel ™ K4M and Methocel™ K15M, are preferred in 1:1 ratio, for the desired sustained release effect. Whereas, the water soluble and/or gellable polymer to be used in the coating, should dissolve immediately in water or should form a weak hydrogel, particularly of low viscosities, such as less than 15cps.

The phrase, “water insoluble or permeable polymer”, as used herein, is intended to encompass a selection from the group comprising methyl/ethyl acrylates, such as Eudragit® RS or RL and Eudragit® NE (manufactured by Rohm Pharma GmbH), cellulose based polymer such as cellulose acetate, cellulose acetate pseudolatex, cellulose acetate propionate, cellulose acetate butyrate, ethyl cellulose (available from Dow chemical company as Ethocel™ 4 cps, Ethocel™ 7 cps, Ethocel™ 10 cps, Ethocel™ 20 cps, Ethocel™ 45 cps, Ethocel™ 100 cps), ethyl cellulose pseudolatex (such as Surelease® as supplied by 10 Colorcon, West Point, Pa. or Aquacoat.™. as supplied by FMC Corporation, Philadelphia, Pa.), nitrocellulose, hydroxyl polyvinyl alcohol-maleic anhydride copolymers, beta-pinene polymers, glyceryl esters of wood resins. A preferred polymer is ethyl cellulose, preferably Ethocel™ 4 cps and/or Ethocel™ 7 cps and/or Ethocel™ 10 cps.

The phrase, “water soluble and/or gellable polymer” as used herein, is intended to encompass a selection from the group comprising of methyl cellulose, hydroxypropyl-methylcellulose, hydroxyethylcellulose, hydroxypropylcellulose, hydroxyethylmethylcellulose, carboxymethylcellulose and sodium carboxymethylcellulose; polyvinylpyrrolidone, polyethylene oxide; and polysaccharides. A preferred polymer is hydroxypropyl methylcellulose (HPMC) and derivatives thereof. HPMC derivatives are available in a low, normal or high viscosity grades. The rate of hydration and extent of gelling capacity of polymer controls a release rate of the drug from the formulation. Specific HPMCs which are most suitable are Methocel™ K100M, K15M, F4M, E4M, K4M, K100LV, K3, E15LV, E15LN, E15CLV, E50, E5 and E3 (available from Dow Chemical, Midland Mich.).

The phrase, “enteric coating polymers” as used herein, is intended to mean a selection from the group comprising cellulose acetate phthalate, hydroxypropylmethylcellulose phthalate, polyvinyl acetate phthalate, carboxymethylethylcellulose, co-polymerized methacrylic acid/methacrylic acid methyl esters such as, for instance, compounds known under the trade name Eudragit® L 12,5 or Eudragit® L 100, Eudragit® L30D-55 (Rohm Pharma) or similar compounds used to obtain enteric coatings. The enteric coating can also be applied using water-based polymer dispersions, e.g. Aquateric®. (FMC Corporation), Eudragit® L100-55 (Rohm Pharma), Coating CE 5142 (BASF). Preferred enteric coating polymer selected from either hydroxypropylmethylcellulose phthalate or Eudragit® L30D-55.

The enteric coating layer can optionally contain a pharmaceutically acceptable plasticizer such as, for instance, cetanol, triacetin, citric acid esters, triethylcitrate, propylene glycol, dibutyl succinate or similar plasticizers. Dispersants such as talc, colorants and pigments may also be included into the enteric coating.

The terms “coating” and “coat”, are used interchangeably herein.

The present invention provides an extended release tablet of paliperidone, comprising a) a core containing paliperidone and at least one water soluble and/or gellable polymer, b) a coating comprising at least one water insoluble or permeable polymer, and a water soluble and/or gellable polymer and optionally an enteric polymer. The present invention provides an extended release tablet of paliperidone, comprising a) a core containing paliperidone and at least one water soluble and/or gellable polymer, b) a coating comprising at least one water insoluble or permeable polymer, and a water soluble and/or gellable polymer c) a coating comprising of an enteric polymer. The present invention provides an extended release composition of paliperidone, comprising a) a core component comprising paliperidone and at least one water soluble and/or gellable polymer b) a first coat comprising a water insoluble or permeable polymer and water soluble and/or gellable polymer c) a second coat comprising water insoluble or permeable polymer and water soluble and/or gellable polymer, wherein the water insoluble or permeable polymer and water soluble and/or gellable polymer of first coat and second coat are the same, however the ratio of the water insoluble or permeable polymer and water soluble and/or gellable polymer in second coat is different from that of the first coat.

The present invention presents an extended release composition of paliperidone, comprising a) a core component comprising paliperidone and at least one water soluble and/or gellable polymer b) a first coat comprising a water insoluble or permeable polymer and water soluble and/or gellable polymer c) a second coat comprising water insoluble or permeable polymer and water soluble and/or gellable polymer, wherein the water insoluble or permeable polymer and water soluble and/or gellable polymer of first coat and second coat are the same, however, the ratio of the water insoluble or permeable polymer and water soluble and/or gellable polymer in second coat is different from that of the first coat.

The present invention presents an extended release composition of paliperidone, comprising a) a core component comprising paliperidone and at least one water soluble and/or gellable polymer b) a first coat comprising a water insoluble or permeable polymer and water soluble and/or gellable polymer c) a second coat comprising water insoluble or permeable polymer and water soluble and/or gellable polymer, wherein the water insoluble or permeable polymer and water soluble and/or gellable polymer of first coat and second coat are the same, however, the ratio of the water insoluble or permeable polymer and water soluble and/or gellable polymer in second coat is different from that of the first coat, further comprising one or more additives such as plasticizers (like triacetin, triethylcitrate, MACROGOL™ 6,000, etc.).

The present invention, as previously described above, presents a composition comprising preferably, the ratio of water insoluble or permeable polymer and water soluble and/or gellable polymer in the first coat is about 40:60. The first coat is about 4% to about 20%, preferably about 8% to about 16%, more preferably about 10% to about 14% by weight of an uncoated core. The first coat comprises ethylcellulose (Ethocel™ 10 cps) in combination with hydroxypropylmethylcellulose (Methocel™ E 5 LV) in a ratio of 40:60.

The present invention, as previously described above, presents a composition comprising the ratio of water insoluble or permeable polymer and water soluble and/or gellable polymer in second coat is about 50:50. The second coat is about 1% to about 15%, preferably about 3% to about 10%, more preferably about 4% to about 8% of an uncoated core. The second coat comprises ethylcellulose (Ethocel™ 10 cps) in combination with hydroxypropylmethylcellulose (Methocel™ E 5 LV) in a ratio of 50:50.

The present invention provides an extended release tablet of paliperidone, comprising a) a core containing paliperidone and at least one water soluble and/or gellable polymer, wherein the at least one water soluble and/or gellable polymer is about 10% to about 40%, preferably about 15% to about 35% by weight of an uncoated core; wherein the uncoated core comprises hydroxypropylmethylcelluloses of a viscosity more than 100 cps, such as Methocel K4M and/or Methocel™ K15M and/or Methocel™ K100M CR, which in turn is about 30% by weight of an uncoated core b) a coating comprising at least one water insoluble or permeable polymer, and a water soluble and/or gellable polymer; wherein the coating is about 4% to about 20%, preferably about 8% to about 16%, more preferably about 10% to about 14% by weight of an uncoated core. The coating comprises ethylcellulose (Ethocel™ 10 cps) in combination with hydroxypropylmethylcellulose (Methocel E 5 LV) which in turn is about 12% by weight of uncoated core.

The present invention provides an extended release tablet of paliperidone, comprising a) core containing paliperidone and at least one water soluble and/or gellable polymer, wherein the at least one water soluble and/or gellable polymer is about 10% to about 40%, preferably about 15% to about 30% by weight of an uncoated core. The uncoated core comprises hydroxypropylmethylcelluloses of a viscosity more than 100 cps, such as Methocel™ K4M and/or Methocel™ K15M and/or Methocel™ K100M CR, which is in turn about 30% by weight of an uncoated core b) a coating comprising at least one water insoluble or permeable polymer, and a water soluble and/or gellable polymer; wherein, the coating is about 4% to about 20%, preferably about 8% to aboutl6%, more preferably about 10% to about 14% by weight of an uncoated core. The coating comprises ethylcellulose (Ethocel™ 10 cps) in combination with hydroxypropylmethylcellulose (Methocel™ E 5 LV) which in turn is about 4.5% by weight of uncoated core ; c) an enteric coat comprising an enteric polymer, which is about 1% to about 20%, preferably about 2% to about 14%, more preferably about 3% to about 10% by weight of an uncoated core. The enteric coat comprises methacrylic acid copolymer (Eudragit® L30D-55) or hydroxypropylmethylcellulose phthalate, which in turn is about 5% by weight of an uncoated core.

The core tablets of present invention optionally contain one or more pharmaceutically acceptable excipients. Useful pharmaceutically acceptable excipients include, but are not limited to diluents, binders, surface active agents, disintegrants, glidants and lubricants and a mixture thereof. The core may be formulated into small beads, pellets, granules, fine granules, mini-tablets or tablets, preferably tablets.

Suitable diluents include, but are not limited to, cellulose-derived materials like powdered cellulose, microcrystalline cellulose (e.g. Avicel®101, Avicel®112 available from FMC BioPolymer, Philadelphia, Pa. USA), microfine cellulose, carboxymethylcellulose salts (such as carboxymethylcellulose calcium) and other substituted and unsubstituted celluloses; starch such as maize starch; pregelatinized starch; lactose, preferably lactose monohydrate (e.g. Pharmatose®, SuperTab® SD, SuperTab® 11SD available from DMV Fonterra excipients); talc; waxes; sugars; sugar alcohols like mannitol and sorbitol; acrylate polymers and copolymers; dextrates; dextrin; dextrose; maltodextrin; pectin; gelatin; inorganic diluents like calcium carbonate, dibasic calcium phosphate dihydrate, tribasic calcium phosphate, calcium sulfate, magnesium carbonate, magnesium oxide, sodium chloride, combine materials like STARLAC and other diluents known to the pharmaceutical industry. More preferred diluents include, lactose monohydrate (SuperTab® 11SD), mannitol or sorbitol.

Suitable binders include, for example, polyvinylpyrrolidone, lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatin and sodium alginate.

Suitable surface active agents include, but are not limited to, sodium lauryl sulphate, polysorbates, decussate sodium, benzalkonium chloride, benzethonium chloride, cetrimide, polyvinyl alcohol and the like and mixtures thereof.

Suitable disintegrants include croscarmellose sodium (e.g. Ac Di Sol.™., Primellose.™.), crospovidone (e.g. Kollidon™, Polyplasdone™), microcrystalline cellulose, polacrilin potassium, powdered cellulose, pregelatinized starch, sodium starch glycolate (e.g. Explotab™, Primoljel™.) and starch. Preferred disintegrants include croscarmellose sodium and microcrystalline cellulose.

A lubricant may be added to the pharmaceutical compositions of the present invention to reduce adhesion and/or ease the release of the product from e.g. the die. Suitable lubricants include, but are not limited to, stearic acid, magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, talc and zinc stearate. Stearic acid and/or magnesium stearate are preferred.

Glidants can be added to improve the flowability of a solid composition before compaction and to improve the accuracy of dosing especially during compaction and capsule filling. Excipients that may function as glidants include colloidal silicon dioxide, magnesium trisilicate, powdered cellulose, and talc. The preferred glidant is colloidal silicon dioxide.

Additional conventional excipients which may be added include preservatives like propyl paraben or methyl paraben and anti-oxidants like butylated hydroxy toluene (BHT).

The film coating, in addition to the coat of “water insoluble or permeable” and “polymer water soluble and/or gellable” polymer as described above, may comprise one or more film-formers or binders, such as a hydrophilic polymer like hydroxypropylmethylcellulose (such as Methocel™ E15LV, HPMC E5LV, HPMC E3LV), Polyvinylypyrollidone (Povidone). The film coat preferably comprises HPMC E3LV and one or more plasticizers, such as polyethylene glycol (MACROGOL 400, MACROGOL 8000 etc), triethylcitrate, Triacetin, diethyl phthalate, propylene glycol, glycerin, butyl phthalate, castor oil, polysorbate 80 and the like.

The coating is applied from a solvent system containing one or more solvents including water, alcohols like methyl alcohol, ethyl alcohol or isopropyl alcohol, ketones like acetone, or ethylmethylketone, chlorinated hydrocarbons like methylene chloride, dichloroethane, and 1,1,1-trichloroethane.

Where a color is employed, the color will be applied together with the film former, plasticizer and solvent composition.

Generally, the ranges of the individual ingredients of the pharmaceutical composition of the present invention are set forth in Table 1.

TABLE 1

| Ingredients | % |
|---|---|
| Diluent | 50-300 |
| Binder | 1-30 |
| Antioxidants | 0.01-5.00 |
| Lubricant | 0.5-8.0 |
| Plasticizer | 0.5-3.0 |
| Surfactant | 0.5-5.0 |

Typically the paliperidone or pharmaceutically acceptable salt thereof will be present in an amount within the range of from about 1 mg to about 15 mg.

The pharmaceutical dosage form of the invention may be prepared, using standard techniques and manufacturing processes generally known in the art, for example by sifting and dry blending the components or by sifting and wet mixing of the components.

The present invention also provides a process for the preparation of an extended release composition comprising a) mixing of paliperidone and at least one water soluble and/or gellable polymer along with diluents to form a core b) applying first coat comprising water insoluble or permeable polymer and a water soluble and/or gellable polymer of a ratio of about 40:60; further c) applying second coat comprising water insoluble or permeable polymer and a water soluble and/or gellable polymer of a ratio of about 50:50.

The following example is provided to enable one skilled in the art to practice the invention and is merely illustrative of the invention. The examples should not be read as limiting the scope of the invention as defined.

### EXAMPLES

### Example 1

Extended Release Tablet comprising Hydroxypropylmethylcellulose (Methocel™K15M) in core and coating with Ethylcellulose (Ethocel™ 10cps) & hydroxypropylmethylcellulose (Methocel™ E5LV).

TABLE 2

| Sr. no. | Intra granular | mg/tab |
|---|---|---|
| 1 | Paliperidone | 6.00 |
| 2 | Microcrystalline cellulose (Avicel ® PH112) | 200.30 |
| 3 | Hydroxypropyl methyl cellulose (Methocel ™ K4M) | 20.00 |
| 4 | Butylated Hydroxy toluene | 0.200 |
| 5 | Hydroxypropyl methyl cellulose (Methocel ™ K15M ) | 30.00 |
| 6 | Aerosil-200 | 1.00 |
| 7 | Magnesium Stearate | 2.50 |
| | Core Tablet weight (mg) | 260.00 |
| | Coating | |
| 8 | Ethyl cellulose (Ethocel 4 cps) | 24.80 |
| 9 | Hydroxypropyl methyl cellulose (Methocel ™ E5LV) | 6.20 |
| 10 | Triethyl citrate | 4.00 |
| 11 | MDC: IPA ^{$} | — |
| | Final Weight (mg) | 294.00 |

### Brief Manufacturing Process:

1. Tablet core is prepared by mixing Paliperidone, Microcrystalline cellulose (Avicel PH 112), Hydroxypropyl methyl cellulose (K4M), Hydroxypropyl methyl cellulose (K15M) and BHT in a mixer for about 10 minutes, adding sieved Colloidal silicon dioxide, Magnesium Stearate to the blend, and mixing for about 5 minutes, and compressing the mixed blend to form tablets in a suitable tablet press.
2. Ethocel 4 CPS, Hydroxypropyl methyl cellulose (E5LV) and Triethyl citrate are dissolved in a mixture of the process solvents.
3. The core tablets of 1 are coated with the solution from 2.

### Example 2

Extended Release Tablet comprising Hydroxypropylmethylcellulose (Methocel™100M) in core and coating in two different layers one of which comprises water insoluble polymer Ethyl cellulose (Ethocel™ 10 cps) and other enteric polymer (Eudragit® L30D-55).

TABLE 3

| Sr. no. | Ingredients | Mg/tablet |
|---|---|---|
| | Intra granular | |
| 1 | Paliperidone | 6.00 |
| 2 | Micr^{oc}rystalline cellulose (Avicel ® PH101) | 190.65 |
| 3 | Hydroxypropyl methyl cellulose(Methocel ™ | 10.00 |
| | E50LV) | |
| 4 | Purified water $ | ^{q.}s |
| | Extra granular | |
| 5 | Hydroxypropyl methyl ^{cel}lulose (Methocel ™ | 37.5 |
| | K100M ) | |
| 6 | Butylated Hydroxy toluene | 2.00 |
| 7 | Aerosil-200 | 1.35 |
| 8 | Magnesium Stearate | 2.5 |
| | Core Tablet weight | 250.0 |
| | Coating (PART A) | |
| 9 | Ethyl cellulose (Ethocel ™ 10 cps) | 9.78 |
| 10 | Hydroxypropyl methyl cellulose (Methocel ™ | 1.09 |
| | E5LV) | |
| 11 | Triethyl citrate | 1.63 |
| 12 | Isopropyl alcohol: Dichloromethane | q.s |
| | Coating (PART B) | |
| 13 | Methacrylic acid Copolymer (Eudragit^{(R)} L30D-55) | 12.08 |
| 14 | Triacetin | 1.81 |
| 15 | Talc | 3.63 |
| 16 | Purified Water | q.s |
| | Final Weight (mg) | 280.00 |

### Brief Manufacturing Process

1. Preparation of a tablets core by mixing the stated amounts of Paliperidone, Microcrystalline cellulose (Avicel PH 112), Hypromellose (HPMC E50LV) in rapid mixer granulator for about 10 minutes; the uniform powder mixture is moistened with purified water and the uniformly moistened mass is dried in a fluidized-air bed at about 40° C. to about 45° C. The dried granulate, milled through 1.5 mm screen and mixed with Hypromellose (K100M) and BHT for about 5 minutes followed by blending it with magnesium stearate and colloidal silica (Aerosil 200) passed through a 60 mesh screen. The granulation blend is then compressed into tablets.
2. Ethocel 10 cps, Hydroxypropyl methyl cellulose (HPMC E5LV) and Triethyl citrate are dissolved in a mixture of Isopropyl alcohol: Dichloromethane.
3. The core tablets of 1 are coated with the solution from 2.
4. Triacetin was dissolved in Purified Water. Talc and Eudragit L30D-55 are added and mixed to form enteric coating dispersion.
5. The coated tablets of 3 are further coated using enteric coating dispersion of 4.

### Example 3

Extended Release Tablet Comprising Hydroxypropyl Methyl Cellulose (Methocel™K4M) & Hydroxypropyl Methyl Cellulose (Methocel™K 15 MCR) in core and first coat of Ethyl cellulose 10 cps (Ethocel™ 10 cps) and Hydroxypropylmethylcellulose (Methocel™ E 5 LV) in ratio of 40:60 by weight and second coat of Ethyl cellulose 10 cps (Ethocel™ 10cps) and Hydroxypropylmethylcellulose (Methocel™ E 5 LV) in ratio of 50:50 by weight.

TABLE 4

| S. No | Ingredients | Mg/tablet |
|---|---|---|
| | Direct Compression | |
| 1 | Paliperidone | 6.00 |
| 2 | Lactose monohydrate (SuperTab ® 11SD) | 235.00 |
| 3 | Hydroxypropyl Methyl Cellulose (Methocel ™ K4M) | 55.00 |
| 4 | Hydroxypropyl Methyl Cellulose (Methocel ™ | 55.00 |
| | K 15 MCR) | |
| 5 | Talc | 3.60 |
| 6 | Magnesium Stearate | 5.40 |
| | Core Tablet weight (mg) | 360.00 |
| | Functional Coating-I | |
| 7 | Ethyl Cellulose (ETHOCEL ™ 10 cps) | 15.68 |
| 8 | Hydroxypropyl Methyl Cellulose (Methocel E 5 LV) | 23.52 |
| 9 | Triethyl Citrate | 4.00 |
| 10 | Isopropyl Alcohol $ | qs |
| 11 | Methylene Chloride $ | qs |
| | Coated Tablet (mg) | 403.20 |
| | Functional Coating-II | |
| 12 | Ethyl Cellulose (ETHOCEL™ 10 cps) | 11.91 |
| 13 | Hydroxypropyl Methyl Cellulose (Methocel ™ E 5 LV) | 11.91 |
| 14 | Triethyl Citrate | 2.38 |
| 15 | Isopropyl Alcohol $ | qs |
| 16 | Methylene Chloride $ | qs |
| | Coated Tablet (mg) | 429.408 |

### Brief Manufacturing Process:

1. Tablet core is prepared by mixing Paliperidone, Lactose monohydrate (Supertab 11SD),

Hydroxypropyl methyl cellulose (K4M), Hydroxypropyl methyl cellulose (K15M) in a blender for about 10 minutes, adding sieved talc followed by the addition of Magnesium Stearate to the blend, and mixing for about 5 minutes, and compressing the mixed blend to form tablets in a suitable tablet press.
2. Ethocel™ 10 cps, Hydroxypropyl methyl cellulose (E5LV) in ratio of 40:60 and Triethyl citrate are dissolved in a mixture of the process solvents
3. The core tablets of 1 are coated with the solution from 2.
4. Ethocel™ 10 cps, Hydroxypropyl methyl cellulose (E5LV) in ratio of 50:50 and Triethyl citrate are dissolved in a mixture of the process solvents
5. The coated tablets of 3 are coated with the solution from 4.

Tablets obtained in example 1, 2 and 3 are further coated with non functional film coating, wherein the non functional film forming polymer selected from low viscosity grade hydroxypropylmethylcellulose (Methocel™ E3, Methocel™ E5) or polyvinylpyrrolidone (KOLLIDON® 30) or Opadry® II Coating Composition available from Colorcon®.

1. An extended release composition of paliperidone suitable for oral administration comprising: (a) a core comprising (i) a therapeutically effective amount of a paliperidone (ii) a water soluble and/or gellable polymer (iii) a diluent, and (iv) a lubricant b) a controlled release water insoluble or permeable polymer coat which surrounds said core; wherein said extended release composition releases paliperidone over a period of about 24 hours.

2. The composition of claim 1, further coated with enteric polymer.

3. The composition of claim 1, wherein the water soluble and/or gellable polymer is selected from the group comprising of methyl cellulose, hydroxypropyl methylcellulose, hydroxyethyl cellulose, hydroxypropyl cellulose, hydroxyethyl methylcellulose, carboxymethylcellulose and sodium carboxymethylcellulose; polyvinylpyrrolidone, polyethylene oxide; and polysaccharides, and derivatives thereof.

4. The composition of claim 1, wherein the diluent is selected from microcrystalline cellulose, microfine cellulose, carboxymethylcellulose salts and other substituted and unsubstituted celluloses; starch such as maize starch; pregelatinized starch; lactose, preferably lactose monohydrate.

5. The composition of claim 1, wherein the lubricant is selected from stearic acid, magnesium stearate, calcium stearate, glyceryl mono stearate, glyceryl palmitostearate, hydrogenated castor oil, hydrogenated vegetable oil, mineral oil, polyethylene glycol, sodium lauryl sulfate, sodium stearyl fumarate, talc and zinc stearate.

6. The composition of claim 1, wherein the water insoluble or permeable polymer is selected from methyl/ethyl acrylates, cellulose acetate, cellulose acetate pseudolatex, cellulose acetate propionate, cellulose acetate butyrate, ethyl cellulose, nitrocellulose, hydroxyl polyvinyl alcohol-maleic anhydride copolymers.

7. The composition of claim 2, wherein the enteric coating polymer is selected from cellulose acetate phthalate, hydroxypropylmethylcellulose phthalate, polyvinyl acetate phthalate, carboxymethylethylcellulose, co-polymerized methacrylic acid/methacrylic acid methyl esters.

8. An extended release tablet of paliperidone, comprising a) a core component comprising paliperidone and at least one water soluble and/or gellable polymer b) a first coat comprising a water insoluble or permeable polymer and water soluble and/or gellable polymer c) a second coat comprising water insoluble or permeable polymer and water soluble and/or gellable polymer, wherein the water insoluble or permeable polymer and water soluble and/or gellable polymer of first coat and second coat are same.

9. The tablet of claim 8 wherein, the water soluble and/or gellable polymer is about 10% to about 40% by weight of an uncoated core.

10. The tablet of claim 8 wherein, the water soluble and/or gellable polymer is about 15% to about 35% by weight of an uncoated core.

11. The tablet of claim 8 wherein the ratio of water insoluble or permeable polymer and water soluble and/or gellable polymer in the first coat is about 40:60 and in the second coat is about 50:50.

12. The tablet of claim 8 wherein, the first coat is about 4% to about 20% by weight of an uncoated core and the second coat is about 1% to about 15% of an uncoated core.

13. The tablet of claim 8 wherein the first coat is about 10% to about 14% by weight of an uncoated core and the second coat is about 4% to about 8% by weight of an uncoated core.

14. The tablet of claim 8, further coated with non-functional film forming polymer.

15. The tablet of claim 14, wherein the non-functional film forming polymer is a low viscosity grade hydroxypropylmethylcellulose.

16. A process for the preparation of an extended release composition of paliperidone suitable for oral administration comprising a) mixing of paliperidone and at least one water soluble and/or gellable polymer along with diluents to form a core b) applying a first coat comprising water insoluble or permeable polymer and a water soluble and/or gellable polymer in a ratio of about 40:60; further c) applying a second coat comprising water insoluble or permeable polymer and a water soluble and/or gellable polymer in a ratio of about 50:50.
